(19) United States
(12) Patent Application Publication
(10) Pub. No.: US 2021/0069439 A1
(43) Pub. Date: Mar. 11, 2021

(54) H-Intubation Blade

(71) Applicant: William Miguel Hasbun, Mt. Laurel, NJ (US)

(72) Inventor: William Miguel Hasbun, Mt. Laurel, NJ (US)

(21) Appl. No.: 17/010,996

(22) Filed: Sep. 3, 2020

Related U.S. Application Data
(60) Provisional application No. 62/973,016, filed on Sep. 11, 2019.

(51) Int. Cl. A61M 16/04; A61B 1/267; A61B 1/00; A61B 1/06

(52) U.S. Cl. CPC A61M 16/0488; A61B 1/0684; A61B 1/00066; A61B 1/267

(57) ABSTRACT

The H-Intubation Blade is a multi-size intubation blade for medical and veterinarian use. The H-Intubation Blade can be used on different sized patients and with the different handles on the market whether they are green in which a light bulb provides the illumination at the base of the handle or traditional handle in which energy is provided from the handle to power a light bulb in the blade itself The H-Intubation Blade changes size by a mounting piece that hooks to the handle and runs back and forth on a locking rail system.

This device is a multi size intubation blade for medical and veterinarian use. The present invention is directed to the health care and veterinarian industry and qualified workers. The current state of the art is a fixed size blade which must be exchanged for a smaller or larger size depending on the size of the patient. The H-Intubation Blade can be used with the different handles whether they are green (in which a light bulb provides the illumination at the base of the handle) or traditional (in which energy is provided from the handle to power a light bulb in the blade itself) which is impossible for the current blades on the market today. The H-Intubation Blade changes size by a mounting piece that hooks to the handle and runs back and forth on a locking rail system.

Description of the drawings in one embodiment of the present invention:

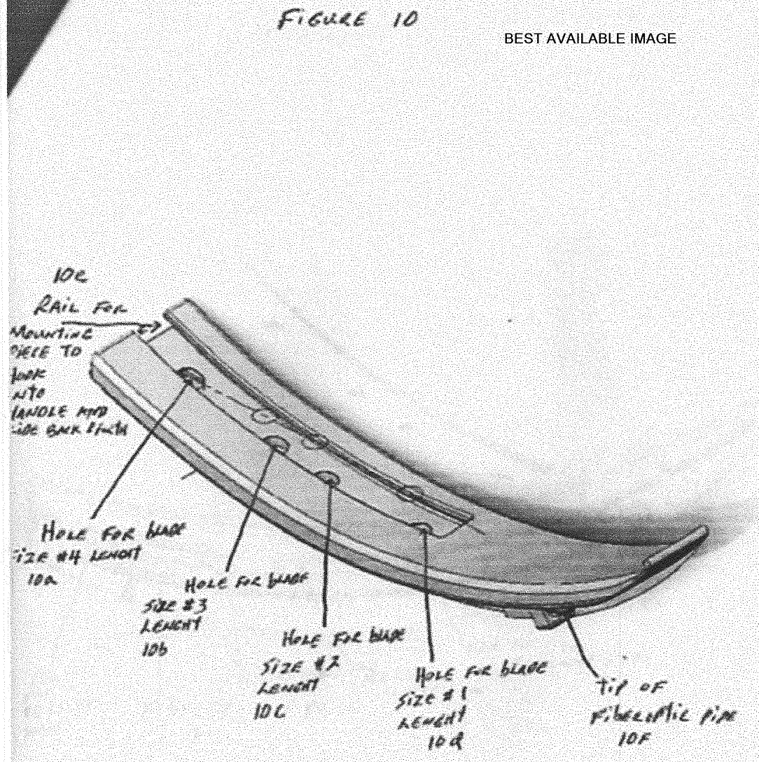

FIG. 10 shows an upper view of the blade.

FIG. 10a shows the pathway hole for the light for size #4 blade.

FIG. 10b shows the pathway hole for the light for size #3 blade.

FIG. 10c shows the pathway hole for the light for size #2 blade.

FIG. 10d shows the pathway hole for the light for size #1 blade.

FIG. 10e shows the rail where the hooking piece runs to adjust (change) the size of the blade.

FIG. 10f shows the fiber optic pipe tip.

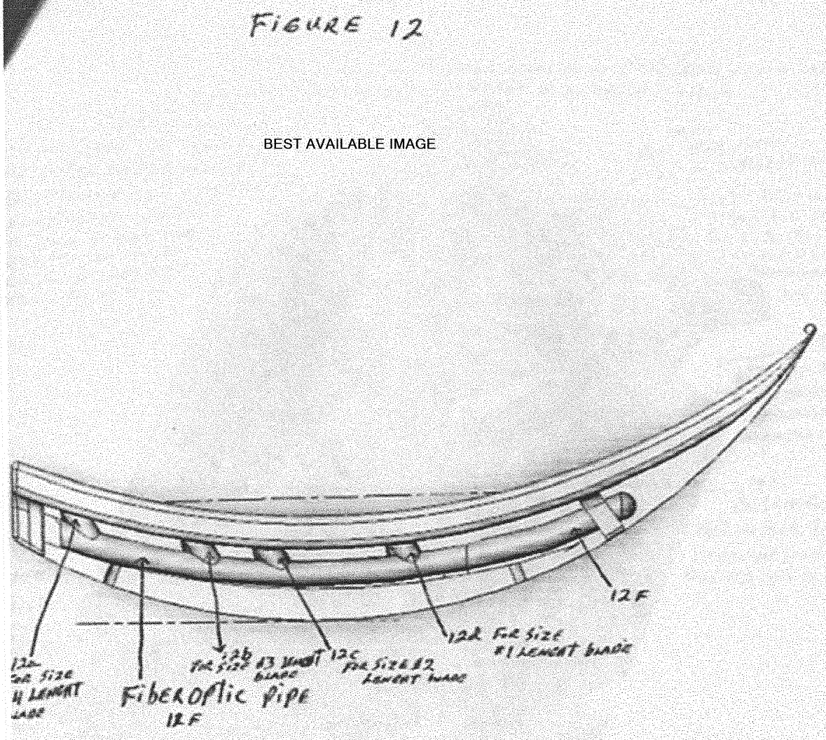

FIG. 12 illustrates a side view of the blade.

FIG. 12a shows the fiber optic for the size #4 blade.

FIG. 12b shows the fiber optic for the size #3 blade.

FIG. 12c shows the fiber optic for the size #2 blade.

FIG. 12d shows the fiber optic for the size #1 blade.

FIG. 12f shows the fiber optic pipe.

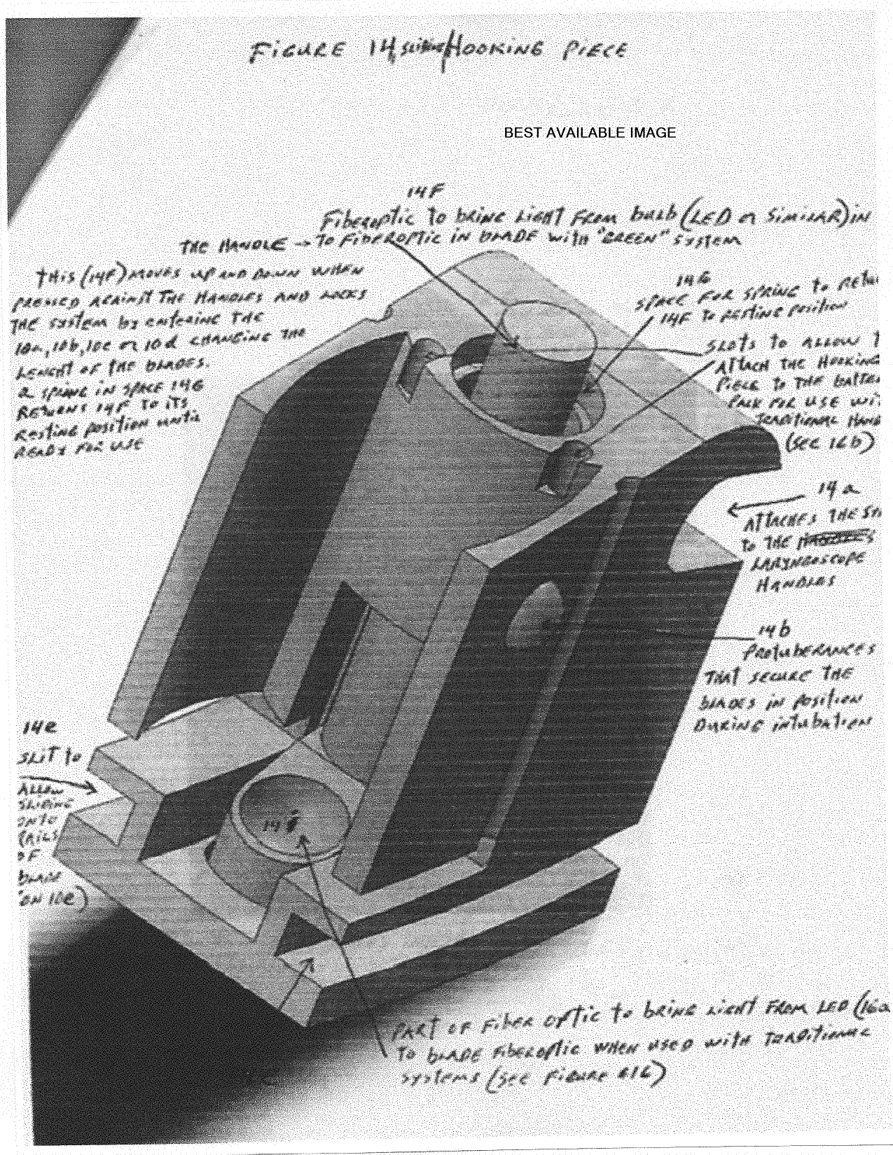

FIG. 14 presents the hooking piece that attaches the blade to the laryngoscope handles.

FIG. 14a is the universal C type of attaching the blades to the handles.

FIG. 14b are half circles that secure the position of the blades once ready for use.

FIG. 14e are the running slits for the rails on the blades.

FIG. 14f is the fiber optics that brings the light from the handle or from 16a to the blade.

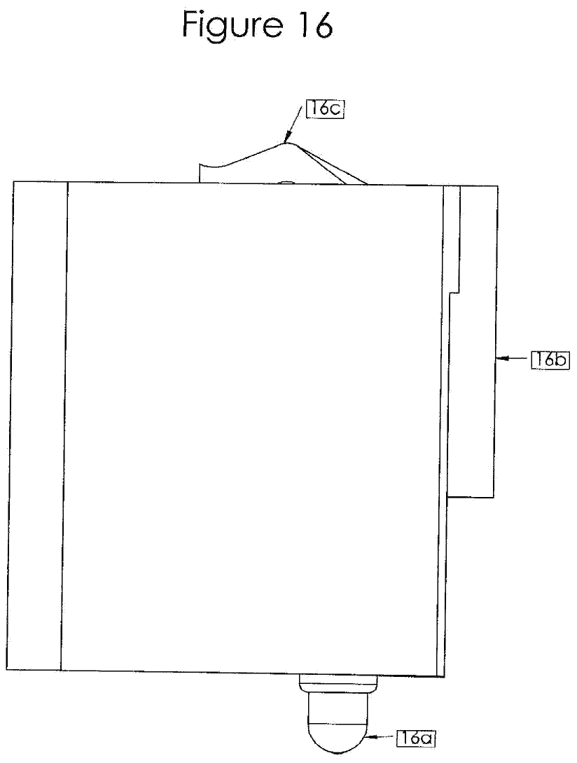

FIG. 16 shows the “power pack” (battery & LED) that powers the laryngoscope when the blade is used with the traditional laryngoscope.

FIG. 16a is the LED bulb.

FIG. 16b hooks the pack to the “sliding/hooking” piece (FIG. 14).

FIG. 16c is the lever that contains the wire to complete the circuit that powers the LED.

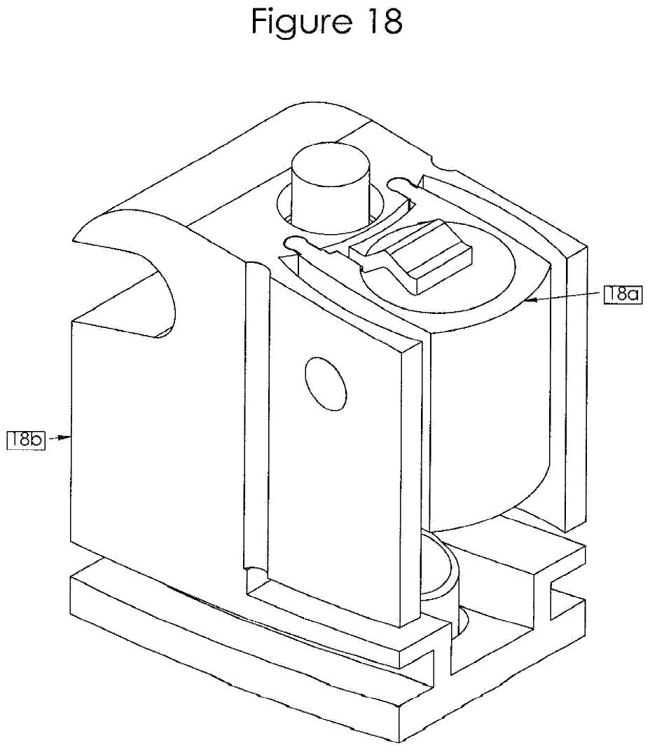

FIG. 18 shows the power pack attached to the “sliding/hooking” piece.

FIG. 18a is the power pack

FIG. 18b is sliding hooking piece

This concept is applicable to both the Macintosh and the Miller type of blades.

1. I claim that this is the only blade that can be used for green type and traditional handles.

2. I claim that this is the only intubation blade that can be used and converted into different sizes without changing the blade.

3. I claim that the light piping of this device is unique since it conveys the light from the source to the tip from at least 4 different positions.
